(19) United States
(12) Patent Application Publication
(10) Pub. No.: US 2026/0198901 A1
(43) Pub. Date: Jul. 16, 2026

(54) Biological Information Acquisition Sensor, Biological Information Acquisition Device, And Biological Information Acquisition Method

(71) Applicant: NOK CORPORATION, Tokyo (JP)

(72) Inventors: Kento TADA, Fujisawa (JP); Toru UDA, Fujisawa (JP); Masayuki TANABE, Kumamoto (JP)

(21) Appl. No.: 19/439,851

(22) Filed: Jan. 5, 2026

(30) Foreign Application Priority Data
Jan. 16, 2025 (JP) 2025-006006

(51) Int. Cl. A61B 8/00; A61B 5/11; A61B 5/296; A61B 5/397; A61B 8/08

(52) U.S. Cl. CPC A61B 8/5284; A61B 5/1107; A61B 5/296; A61B 5/397; A61B 8/085; A61B 8/463; A61B 8/5207

(57) ABSTRACT

In order to confirm an activity state of a pelvic floor muscle during practice of a pelvic floor muscle exercise, a sensor sheet is attached to a region of an abdomen corresponding to a transverse abdominal muscle, and acquires an electromyogram of the transverse abdominal muscle in which a cooperative contraction with the pelvic floor muscle occurs. Here, even when the transverse abdominal muscle is contracted, the pelvic floor muscle is not necessarily contracted, so that the activity state of the pelvic floor muscle is confirmed by an echo. More specifically, an ultrasound sensor is pressed against the abdomen so that the ultrasound probe is brought into contact with the sensor sheet, and an image (M-mode image) of an ultrasound echo indicating an activity state of a bottom of a bladder that can be regarded as the contraction of the pelvic floor muscle is acquired. An exerciser of pelvic floor muscle training acquires a trick of contracting the pelvic floor muscle, that is, an understanding of which part of the body to focus on and how to direct awareness in order to achieve contraction of the pelvic floor muscle.

### CROSS REFERENCE TO RELATED APPLICATION

The present application is based on Japanese Priority Document JP2025-6006 filed on Jan. 16, 2025, the content of which is incorporated herein by reference.

### BACKGROUND OF THE INVENTION

### Field of the Invention

The present disclosure relates to a biological information acquisition sensor, a biological information acquisition device, and a biological information acquisition method.

### Discussion of the Background

Pelvic floor muscle training, which has attracted attention in recent years, is said to originate from the Kegel exercise advocated in the 1940s by Dr. Arnold Kegel, an American obstetrician-gynecologist. Kegel devised the Kegel exercise as part of the treatment for female patients suffering from symptoms of urinary incontinence.

The pelvic floor muscle is a muscle located at the bottom of the pelvis (see FIGS. 1A to 1C) and serves a role of supporting the organs within the pelvis. The pelvic floor muscle group, centered around the pelvic floor muscle, includes the urethral sphincter, and it is considered that when a woman experiences childbirth, the pelvic floor muscle group becomes loosened during delivery, thereby weakening the sphincter function and resulting in cases of urinary incontinence. The pelvic floor muscle training enhances the sphincter function by strengthening the pelvic floor muscle, thereby improving urinary incontinence.

The pelvic floor muscle training not only improves urinary incontinence but also provides functional training of deep trunk muscles, and is therefore effective for maintaining posture and alleviating lower back pain. The pelvic floor muscle training is expected to yield meaningful results in extending healthy life expectancy and in maintaining and improving quality of life (QOL).

On the other hand, there is a trick to the pelvic floor muscle training, and it is difficult for an individual to perceive whether the pelvic floor muscle is actually being trained. Moreover, the pelvic floor muscle is an inner muscle, and a shape change during training cannot be externally confirmed. Therefore, instructors or practitioners of the pelvic floor muscle training cannot know the state of the pelvic floor muscle during training and cannot be certain of the effectiveness of the training.

In such a background, a biofeedback technique that visualizes an activity level of the pelvic floor muscle during training is being studied. For example, see: Kazumi Tsujino and Satoko Hoshino, “Study on Breathing Method for Effective Contraction of Pelvic Floor Muscle: As a Practical Approach to Exercise Instruction for General Middle-Aged and Elderly Women”, Japan Health Promotion Fitness Foundation, 2017, pp. 79 to 90.

In this document (hereinafter, referred to as Tsujino et al.), it is shown that the activity level of the pelvic floor muscle is confirmed from an electromyogram based on a myoelectric potential signal collected from a subject, together with a method of the pelvic floor muscle training, which is referred to as the HA breathing method and the HU breathing method in a lateral recumbent position (see p. 82, “2-4. Measurement Item and Measurement Method”).

As another example of the biofeedback technique, Japanese Patent Application Laid-Open No. 2022-120842 (hereinafter, referred to as Kirino et al.) describes an invention in which an echo image of the bladder is acquired using an ultrasound probe (2) and is displayed on a touch panel screen (11) of a mobile information terminal (1) (see paragraphs 0040 to 0042, 0104, and 0130). Kirino et al. present a method of confirming contraction of the pelvic floor muscle from a change in the size of the bladder (see paragraph 0129).

In Tsujino et al., measurement of the myoelectric potential of the pelvic floor muscle is performed by inserting a dedicated probe into the vagina of a woman. That is, the electromyogram of the pelvic floor muscle is acquired as an intravaginal electromyogram. Since the intravaginal electromyogram reflects the influence of contraction of the perineal membrane region, including the vaginal sphincter and the surrounding external urethral sphincter and the external anal sphincter, as well as contraction of the pelvic diaphragm, the intravaginal electromyogram can be used as a measurement indicator of a muscle activity level of the pelvic floor muscle.

However, it is considered that there is a psychological resistance to collecting data of the intravaginal electromyogram to measure the activity level of the pelvic floor muscle. In addition, it is necessary to be careful about the gender of the instructor assisting with the measurement work, and the measurement cannot be performed anywhere. Moreover, the measurement method can be used only for women and cannot be applied to men.

In this regard, Tsujino et al. mention the relationship between contraction of the transverse abdominal muscle and the rectus abdominis muscle (see FIG. 2) and contraction of the pelvic floor muscle, and examine the relationship between the measurement results of the myoelectric potentials of the pelvic floor muscle and the transverse abdominal muscle and the rectus abdominis muscle (hereinafter, referred to as “transverse abdominal muscle and the like”) (see pp. 82 to 89). Since the myoelectric potential of the transverse abdominal muscle and the like can be measured on the body surface regardless of gender, the psychological resistance is expected to be significantly reduced.

Regarding the essential relationship between the contraction of the pelvic floor muscle and the transverse abdominal muscle and the like, Tsujino et al. have confirmed that, during execution of a rhythmic contraction task accompanied by voluntary contraction of the pelvic floor muscle, the pelvic floor muscle and the transverse abdominal muscle and the like cooperate with each other to maintain the muscle activity (see p. 88, “3-8” and “3-8-1”). For “voluntary contraction of pelvic floor muscle” and “rhythmic contraction task”, see pp. 80 and 81, “2-3-1” to “2-3-3”.

On the other hand, Tsujino et al. have also confirmed that, even during execution of a rhythmic contraction task accompanied by voluntary contraction of the pelvic floor muscle, there are cases in which coordinated contraction between the pelvic floor muscle and the transverse abdominal muscle and the like is not observed (see p. 88, “3-8-2”). Therefore, although evaluating the electromyogram of the transverse abdominal muscle and the like during the training of the pelvic floor muscle is one method for estimating the muscle activity of the pelvic floor muscle, activity of the transverse abdominal muscle and the like does not necessarily indicate activation of the pelvic floor muscle, and it is known that the muscle activity of the pelvic floor muscle cannot be completely estimated from the electromyogram of the transverse abdominal muscle and the like.

Echo images as shown in Kirino et al. faithfully reproduce internal movements of the human body, and are therefore highly reliable as derived feedback indicating the muscle activity of the pelvic floor muscle. On the other hand, in order to acquire echo images, it is necessary to hold an ultrasound sensor with a relatively large and bulky ultrasound probe incorporated therein and press the ultrasound sensor against the abdomen, resulting in a considerable operational burden. Moreover, since the ultrasound sensor needs to be pressed against the abdomen, continuous use causes discomfort. Echo images are not suitable for temporally continuous evaluation.

As described above, the evaluation method based on the electromyogram and the evaluation method using the echo image have been described as the evaluation method for confirming the effectiveness of the pelvic floor muscle exercise in real time. However, both evaluation methods have advantages and disadvantages and are not decisive. It is desired to improve the accuracy of evaluation while using a simple evaluation method based on the electromyogram of the transverse abdominal muscle and the like as a basis.

An object of the present disclosure is to easily and accurately grasp an activity state of a pelvic floor muscle during execution of a pelvic floor muscle exercise.

### SUMMARY OF THE INVENTION

An aspect of a biological information acquisition sensor includes: a sensor sheet that is attached to a human body and acquires an electric signal generated by a muscle; and an ultrasound sensor including an ultrasound probe configured to be pressed on the sensor sheet attached to the human body, the ultrasound sensor receiving a signal of an ultrasound echo from the human body, in which the sensor sheet includes a sheet made of an elastomer, a plurality of electrodes provided on the sheet and made of an elastomer to which conductivity is imparted, a plurality of wiring lines that are respectively connected to the electrodes, provided on the sheet, and made of an elastomer to which conductivity is imparted, and an insulating layer made of an elastomer and fixed to the sheet to cover the wiring lines while leaving portions of the electrodes.

An aspect of a biological information acquisition device includes: the biological information acquisition sensor; electromyogram generation unit that generates image data of an electromyogram based on an electric signal extracted from an output end of the wiring line included in the sensor sheet; and an echo image generation unit that generates, based on a signal of an ultrasound echo output by the ultrasound probe included in the ultrasound sensor, image data of the ultrasound echo.

An aspect of a biological information acquisition method is a biological information acquisition method using the biological information acquisition sensor, the biological information acquisition method including: attaching the sensor sheet to a region of an abdomen corresponding to a transverse abdominal muscle; and pressing the ultrasound sensor against the abdomen so that the ultrasound probe is brought into contact with the sensor sheet attached to the human body.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIG. 1A is a schematic view of a back side of a human body showing a position of a pelvic floor muscle group;

FIG. 1B is a side view of a human body showing a position and a shape of a pelvic floor muscle of a male;

FIG. 1C is a side view of a human body showing a position and a shape of a pelvic floor muscle of a female;

FIG. 2 is a schematic view of a front side of the human body showing a muscle group including a transverse abdominal muscle;

FIG. 3 is a schematic view showing an exerciser of pelvic floor muscle training in a lateral recumbent position as an example of a posture when training the pelvic floor muscle;

FIG. 4 is a schematic view showing an aspect in which a biological information acquisition sensor is attached to the exerciser of the pelvic floor muscle training in a lateral recumbent position and a biological information acquisition method is performed;

FIG. 5 is a plan view of a sensor sheet;

FIG. 6 is a cross-sectional view taken along line A-A in FIG. 5;

FIG. 7 is a plan view of the sensor sheet with an insulating layer removed;

FIG. 8 is a block diagram showing an example of an electromyogram generation unit;

FIG. 9 is a block diagram showing an example of an echo image generation unit;

FIG. 10 is a block diagram showing another example of the electromyogram generation unit;

FIG. 11 is a block diagram showing another example of the echo image generation unit;

FIG. 12 is a block diagram showing another example of an image generation unit (the electromyogram generation unit and the echo image generation unit);

FIG. 13 is a block diagram showing still another example of the image generation unit (the electromyogram generation unit and the echo image generation unit);

FIG. 14A is a schematic view showing a configuration example of each unit in an experiment (comparative experiment) in which the sensor sheet is not used as an experimental example using a phantom and an M-mode image in association with each other;

FIG. 14B is a schematic view showing a configuration example of each unit in an experiment (verification experiment) in which the sensor sheet is used as an experimental example using a phantom and an M-mode image in association with each other;

FIG. 15A is a schematic view showing an echo image obtained in the comparative experiment;

FIG. 15B is a schematic view showing an echo image obtained in the verification experiment;

FIG. 16 is a graph showing a waveform of echo intensities obtained in the experiments shown in FIGS. 14A and 14B.

FIG. 17A is a schematic view showing an echo image at the time of muscle relaxation obtained in the comparative experiment as an example of an experiment using a human body;

FIG. 17B is a schematic view showing an echo image at the time of muscle contraction obtained in the comparative experiment as an example of an experiment using a human body;

FIG. 17C is a schematic view showing an echo image at the time of muscle relaxation obtained in the verification experiment as an example of an experiment using a human body;

FIG. 17D is a schematic view showing an echo image at the time of muscle contraction obtained in the verification experiment as an example of an experiment using a human body; and

FIG. 18 is a graph showing an electromyogram image obtained in the verification experiment with the muscle contraction timing of FIG. 17B and FIG. 17D superimposed thereon.

### DETAILED DESCRIPTION OF THE PREFERRED EMBODIMENTS

Embodiments will be described based on the drawings according to the following items.
1. Overview
2. Evaluation by Electromyogram
   (1) Sensor Sheet
   (2) Electromyogram Generation Unit
3. Evaluation by Echo Image
   (1) Ultrasound Sensor
   (2) Echo Image Generation Unit
4. Biological Information Acquisition Device
5. Biological Information Acquisition Method
   (1) First Step
   (2) Second Step
   (3) Summary
6. Another Example of Image Generation Unit
   (1) Electromyogram Generation Unit
   (2) Echo Image Generation Unit
   (3) Display Aspect
7. Another Example of Image Generation Unit
8. Still Another Example of Image Generation Unit
9. Modification Examples

### 1. Overview

In the present embodiment, a biological information acquisition sensor, a biological information acquisition device, and a biological information acquisition method are disclosed. An object of the biological information acquisition sensor according to the present embodiment, and the biological information acquisition device and the biological information acquisition method using the same is to acquire a contraction amount of a pelvic floor muscle as biological information in order to confirm an activity level of the pelvic floor muscle when performing pelvic floor muscle training.

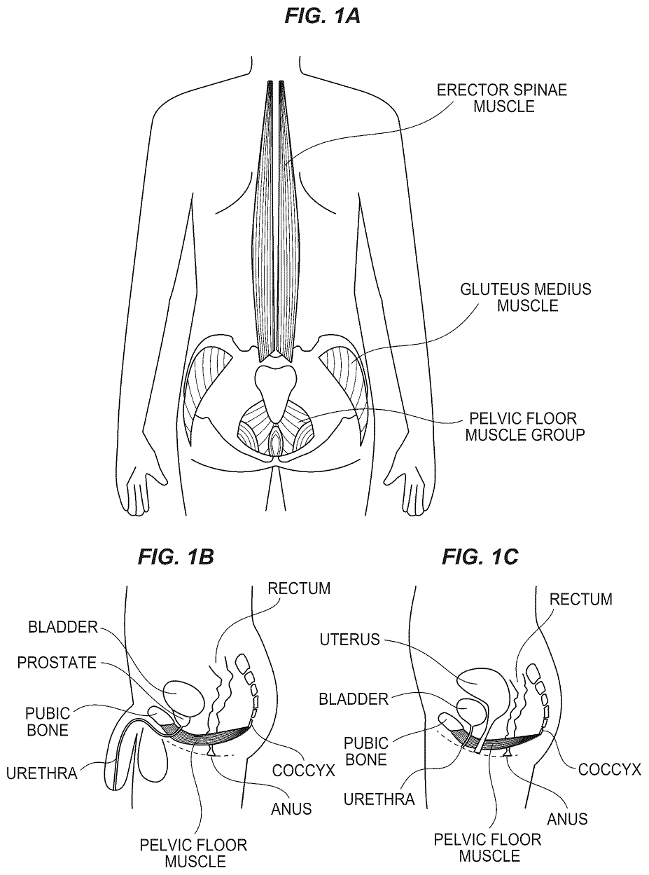

As shown in FIG. 1, the pelvic floor muscle is a muscle located at the bottom of the pelvis and supports the organs within the pelvis (see FIGS. 1A to 1C), and it is difficult to directly acquire a contraction amount thereof. Therefore, in the present embodiment, the contraction amount of the pelvic floor muscle is estimated by an electromyogram obtained by measuring a myoelectric potential of the transverse abdominal muscle, which has been confirmed to have a cooperative contraction with the pelvic floor muscle, that is, an electric signal generated by the muscle (see p. 88, “3-8-1” in Tsujino et al.).

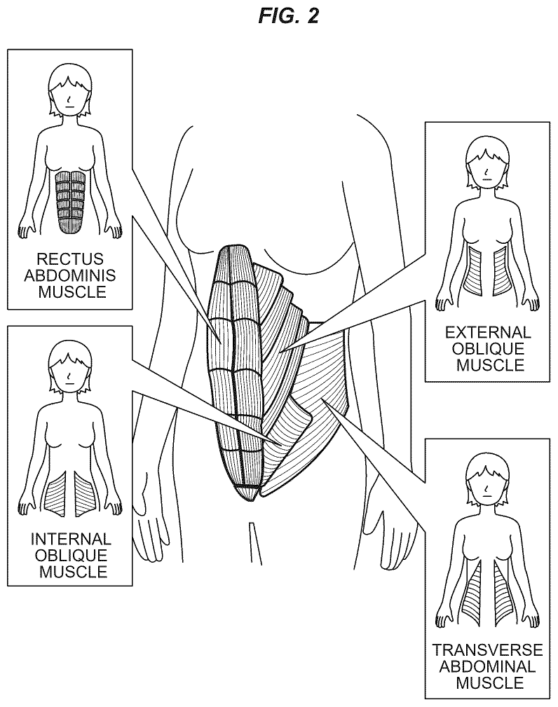

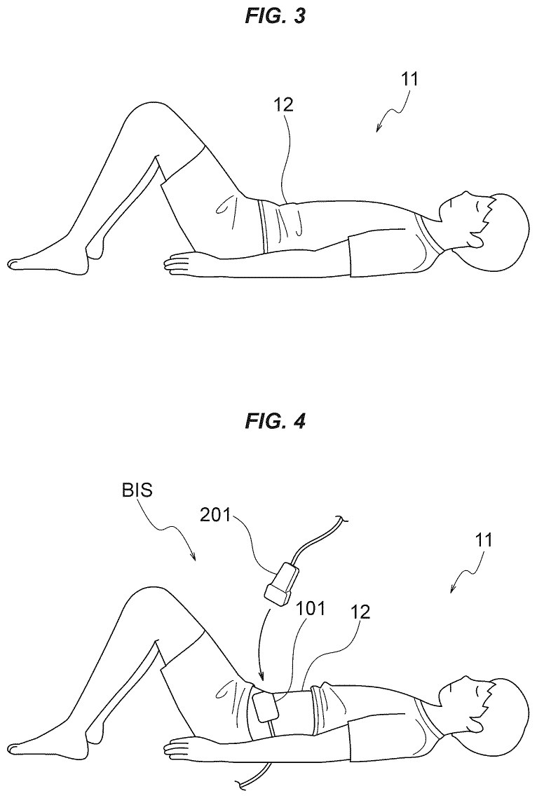

As shown in FIG. 2, the transverse abdominal muscle is a muscle located inside the internal oblique muscle. The myoelectric potential of the transverse abdominal muscle can be acquired by an electrode attached to a body surface with respect to an abdomen 12 of a human body 11 (see FIGS. 2 and 3).

The pelvic floor muscle training is performed, for example, as shown in FIG. 3, using the HA breathing method in a posture of a lateral recumbent position. The HA breathing method is a breathing method in which the lower transverse abdominal muscle is mainly contracted (see “(1) HA breathing method” on p. 79 and p. 81 in Tsujino et al.).

As shown in FIG. 4, in the present embodiment, in order to extract the myoelectric potential of the transverse abdominal muscle, a sensor sheet 101 is attached to a region of the abdomen 12 of the human body 11 corresponding to the transverse abdominal muscle. The sensor sheet 101 functions as a biological information acquisition sensor BIS, and the myoelectric potential of the transverse abdominal muscle acquired by the sensor sheet 101 is converted into image data of an electromyogram by electromyogram generation units 131A to 131D and is displayed on a monitor 172 as an electromyogram (see FIG. 8).

On the other hand, as described above, it has also been confirmed that the cooperative contraction may not occur between the pelvic floor muscle and the transverse abdominal muscle (see p. 88, “3-8-2” in Tsujino et al.). Therefore, a muscle activity of the pelvic floor muscle cannot be completely estimated only from the electromyogram of the transverse abdominal muscle.

Therefore, in the present embodiment, biological information is acquired by an ultrasound echo and is used as appropriate, and a degree of contraction of the pelvic floor muscle can be visually confirmed. However, an image that is visualized as an echo image is not the contraction of the pelvic floor muscle itself but a video of the bladder (see FIGS. 1B and 1C). Since the bottom of the bladder is linked to the contraction of the pelvic floor muscle, the echo image of the bladder can be regarded as the contraction of the pelvic floor muscle.

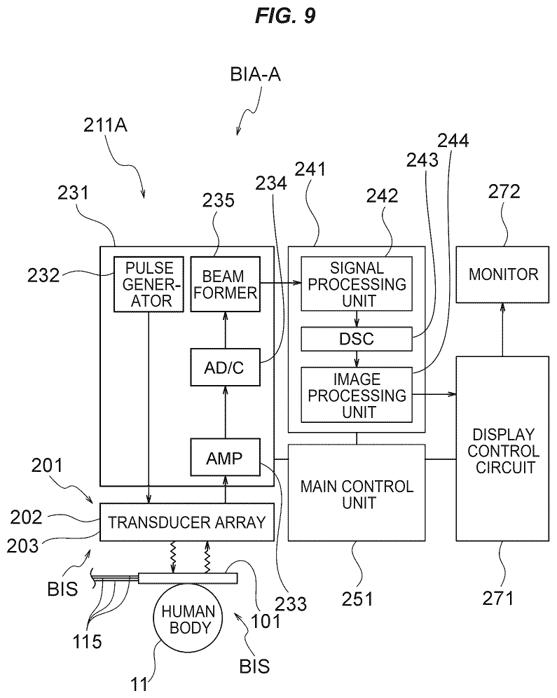

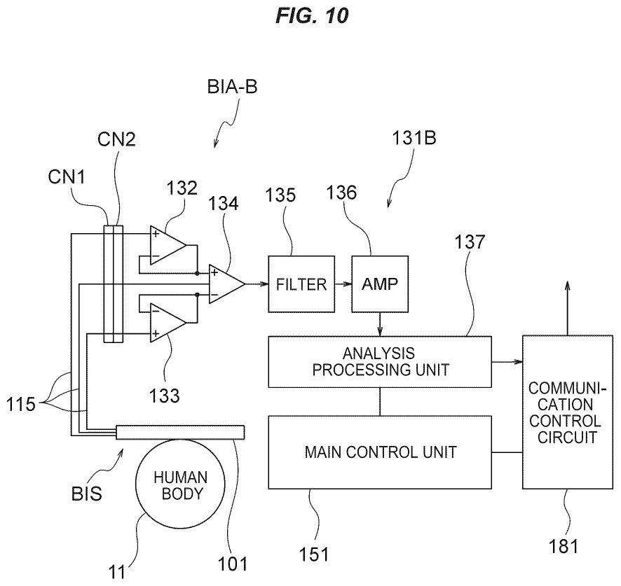

As shown in FIG. 4, in order to acquire the echo image of the bottom of the bladder that can be regarded as a contraction image of the pelvic floor muscle, an ultrasound probe 202 (see FIG. 9) incorporated in an ultrasound sensor 201 is pressed against the abdomen 12 of the human body 11. The ultrasound sensor 201 functions as the biological information acquisition sensor BIS, and a signal of an ultrasound echo acquired by the ultrasound probe 202 is converted into image data of the ultrasound echo by echo image generation units 211A to 211D and is displayed on a monitor 272 as an echo image (see FIG. 9).

In the biological information acquisition method according to the present embodiment, a first step of preparing the sensor sheet 101 and attaching the sensor sheet 101 to the region of the abdomen 12 corresponding to the transverse abdominal muscle and a second step of pressing the ultrasound sensor 201 against the abdomen 12 are executed. In the second step, in order to align the ultrasound probe 202 with a position of the bladder, the ultrasound sensor 201 is pressed against the abdomen 12 so that the ultrasound probe 202 is brought into contact with the sensor sheet 101 attached to the human body 11.

According to the present embodiment, since the ultrasound sensor 201 is pressed against the sensor sheet 101 and the signal of the ultrasound echo is acquired by the ultrasound probe 202 in this state, the sensor sheet 101 is required to have a specific structure. The structure of the sensor sheet 101 will be described in detail later.

In actual operation, in order to monitor an activity state of the pelvic floor muscle, the sensor sheet 101 is attached to the abdomen 12 to monitor an activity state of the transverse abdominal muscle. Here, even when the transverse abdominal muscle is active, the pelvic floor muscle may not be sufficiently contracted. Therefore, the echo image of the bladder acquired by the ultrasound sensor 201 is appropriately monitored, and the activity state of the pelvic floor muscle is confirmed from the visualized state of the bottom of the bladder. As a result, an instructor or exerciser of the pelvic floor muscle training can know the state of the pelvic floor muscle during the training.

As a result, the instructor can provide appropriate advice to the exerciser of the pelvic floor muscle training. The exerciser can obtain feedback on whether or not the training is effectively performed, and by accumulating this experience, the exerciser can acquire a trick of contracting the pelvic floor muscle, that is, an understanding of which part of the body to focus on and how to direct awareness in order to achieve contraction of the pelvic floor muscle.

### 2. Evaluation by Electromyogram

As described above, the configuration required for generating the electromyogram includes the sensor sheet 101 as the biological information acquisition sensor BIS and the electromyogram generation units 131A to 131D. The electromyogram generation unit according to the present embodiment is denoted by reference numeral 131A, and the electromyogram generation units according to the other three embodiments described later are indicated by reference numerals 131B to 131D, respectively.

### (1) Sensor Sheet

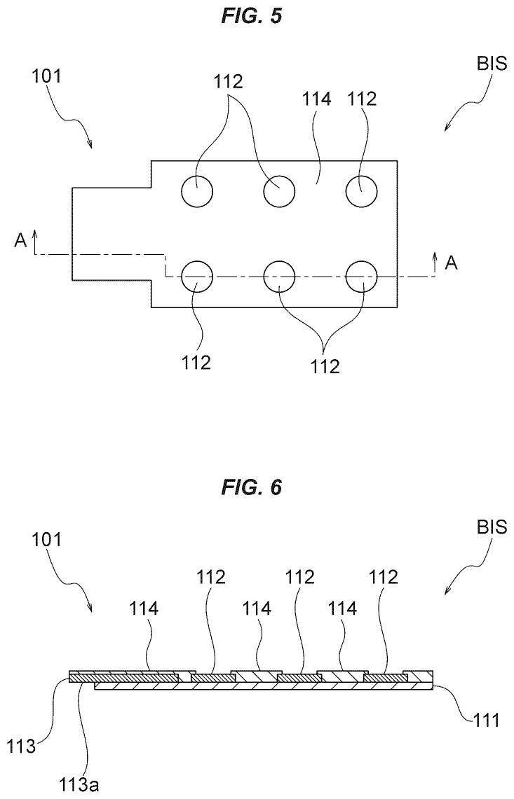

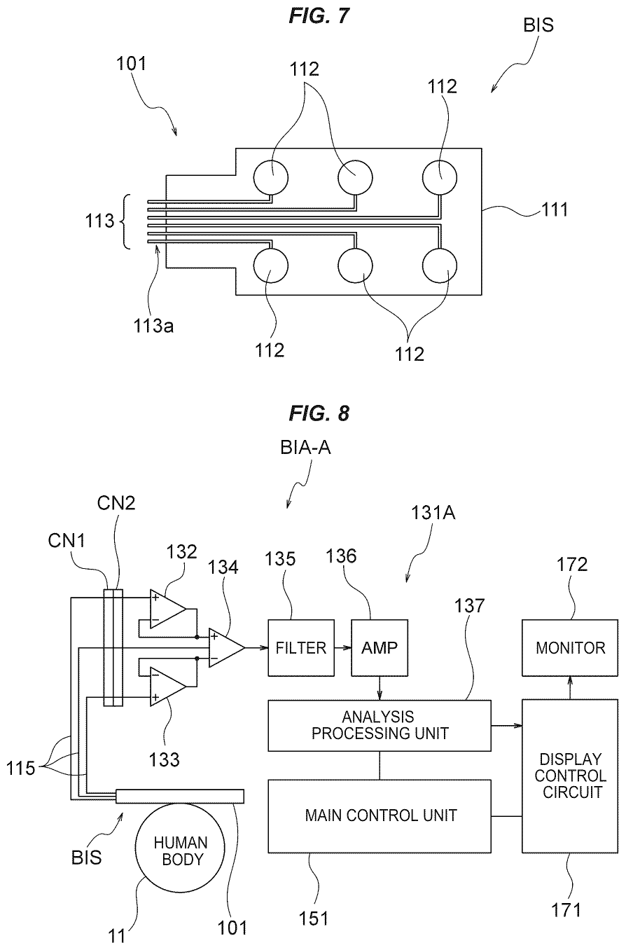

As shown in FIGS. 5 to 7, the sensor sheet 101 is provided with six electrodes 112 on a sheet 111 having a rectangular shape. The electrodes 112 are arranged three on one side and three on the other side at both side ends of the sheet 111 in a width direction.

As shown in FIG. 6 taken along line A-A in FIG. 5, the electrodes 112 and wiring lines 113 are laminated on the sheet 111 in the same layer, and an insulating layer 114 is further laminated to cover peripheral portions of the electrodes 112 and the wiring lines 113.

The sheet 111, the electrodes 112, the wiring lines 113, and the insulating layer 114 are all generated using an elastomer as a material. As the elastomer, for example, a urethane-based elastomer is used. The elastomer is not limited to the urethane-based elastomer, and various elastomers can be appropriately used as long as the elastomer has a property of propagating ultrasound generated by the ultrasound probe 202 incorporated in the ultrasound sensor 201. Examples of the elastomer include a polyester elastomer, a styrene-based elastomer, a polyolefin-based elastomer, and a polyamide-based elastomer.

The sheet 111 is a member that forms a base portion for the entire assembly, and has a stepped shape in which a width of a region in which the wiring lines 113 are extended is narrower than a region in which the electrodes 112 are disposed. A thickness of the sheet 111 is 50 μm or less, for example, 10 to 25 μm or less. Therefore, the sheet 111 has not only stretchability and flexibility but also conformability to an object such as the human body 11.

The electrode 112 has a true circular shape. The wiring line 113 has a linear shape in which one end side is connected to the electrode 112. An end portion of the wiring line 113, which is located on a side opposite to a connection end to the electrode 112, protrudes from an end portion of the sheet 111. The protruding portion is an output end 113a that is connected to a connection line 115 described later by a coupler (not shown) or the like.

Conductivity is imparted to the electrodes 112 and the wiring lines 113 having such a shape. The conductivity is imparted, for example, by forming the electrodes 112 and the wiring lines 113 using a conductive composite material in which conductive particles or conductive fibers are dispersed in an elastomer. As another example, conductivity may also be imparted by forming the electrodes 112 and the wiring lines 113 using an organic conductive polymer compound.

The electrodes 112 and the wiring lines 113 are formed on the sheet 111 by a printing method such as screen printing, ink jet printing, gravure printing, or offset printing. Therefore, the electrodes 112 and the wiring lines 113 have a sufficiently small thickness and, like the sheet 111, have stretchability and flexibility.

The insulating layer 114 is a member having a thickness of 50 μm or less, for example, 10 to 25 μm or less as a thickness that can maintain an insulating property against the wiring lines 113. As in the sheet 111, the insulating layer 114 has not only stretchability and flexibility but also conformability to an object such as the human body 11.

The insulating layer 114 not only covers one surface side of the sheet 111 on which the electrodes 112 and the wiring line 113 are formed, but also protrudes from the sheet 111 at the output end 113a of the wiring line 113 to support the output end 113a. In a predetermined region including a portion that supports the output end 113a, the insulating layer 114 may be formed of a separate member having higher rigidity.

The sensor sheet 101 configured as described above is attached to the human body 11 so that a surface of the sensor sheet 101 on an insulating layer 114 side on which the electrodes 112 are exposed comes into contact with the body. Here, the sensor sheet 101 is thin enough to be able to follow a shape of the object such as the human body 11, not only in terms of each part of the sheet 111 and the insulating layer 114 but also as a whole. Therefore, the sensor sheet 101 maintains the conformability to the human body 11 including the abdomen 12. However, if necessary, the surface (upper surface side in FIG. 6) that comes into contact with the human body 11 may be provided with an adhesive layer (not shown) having a property of propagating ultrasound and an insulating property as necessary.

FIG. 6 showing a cross-sectional shape of the sensor sheet 101 depicts the thickness of each part in an exaggerated manner. Therefore, referring to FIG. 6, the surface of the electrode 112 is disposed at a position considerably recessed from the surface of the insulating layer 114, and the electrode 112 may appear as if the surface of the electrode 112 does not come into contact with the human body 11 at all or comes into contact with the human body 11 only with a small area. Contrary to this, since the thickness of the insulating layer 114 is 50 μm or less, for example, 10 to 25 μm or less, a thickness dimension of the insulating layer 114 from the surface of the electrode 112 is extremely small and does not interfere with the contact of the electrode 112 with the human body 11. When the sensor sheet 101 is attached to the human body 11, the six electrodes 112 come into contact with the body with a sufficient area.

Regarding the sensor sheet 101 described above, related art is described in Japanese Patent Application Laid-Open No. 2019-051236 (hereinafter, referred to as Araki et al.). However, Araki et al. describes only an electrode sheet formed to be thin using an elastomer as a material. No disclosure or suggestion of the property of propagating ultrasound is shown in Araki et al.

### (2) Electromyogram Generation Unit

As shown in FIG. 8, the electromyogram generation unit 131A is connected to the sensor sheet 101 by connectors CN1 and CN2.

The connector CN1 is a connector on a sensor sheet 101 side, and is connected to the wiring lines 113 by the connection line 115. The electrodes 112 provided on the sensor sheet 101 are provided in two rows, each row including a set of three electrodes. Here, for simplicity of the description, a set of three electrodes in one row is taken as an example, and the connection to the electromyogram generation unit 131A is described.

A set of electrodes 112 includes a first electrode, a second electrode, and a body ground, which have different roles. As an example, in FIG. 5, the first electrode, the body ground, and the second electrode are arranged in order from the right, and in FIG. 8, the first electrode, the body ground, and the second electrode are arranged in order from the top.

Therefore, from the connector CN2 on an electromyogram generation unit 131A side connected to the connector CN1, the wiring lines corresponding to the first electrode, the body ground, and the second electrode are led out in order from the top in FIG. 8.

The electromyogram generation unit 131A includes two buffer amplifiers 132 and 133 and one differential amplifier 134. The first electrode led out from the connector CN2 is connected to a positive terminal of the buffer amplifier 132, and a negative terminal and an output end of the buffer amplifier 132 are connected to a positive terminal of the differential amplifier 134. The second electrode led out from the connector CN2 is connected to a positive terminal of the buffer amplifier 133, and a negative terminal and an output end of the buffer amplifier 133 are connected to a negative terminal of the differential amplifier 134. The body ground is connected to a ground terminal of the differential amplifier 134.

A circuit including the two buffer amplifiers 132 and 133 and the one differential amplifier 134 outputs a signal of a myoelectric potential in a bipolar induction method. More specifically, a difference in potential between the first electrode and the body ground and a difference in potential between the second electrode and the body ground are obtained, and a difference between the difference in potential generated in the first electrode and the difference in potential generated in the second electrode is output from an output end of the differential amplifier 134 as the signal of the myoelectric potential, that is, an electric signal generated by a muscle.

The output signal of the differential amplifier 134 is filtered by a filter 135, amplified by an amplifier 136, and input to an analysis processing unit 137.

The filter 135 includes a low-pass filter or a high-pass filter, and removes unnecessary low or high frequencies from the output signal of the differential amplifier 134. The amplifier 136 amplifies the signal that has passed through the filter 135 and outputs the amplified signal to the analysis processing unit 137.

The analysis processing unit 137 performs digital conversion on the signal output from the amplifier 136 and performs various types of processing to generate image data of the electromyogram. Therefore, it can be said that the analysis processing unit 137 is a circuit that is a core of the electromyogram generation unit 131A that executes image generation processing based on the electric signal extracted from the output end 113a of the wiring lines 113 of the sensor sheet 101 to generate the image data of the electromyogram.

The electromyogram generation unit 131A includes a main control unit 151. The main control unit 151 is configured by, for example, a microcomputer that interprets and executes a program or an integrated circuit that sequentially executes a specified process, and is responsible for controlling the analysis processing unit 137 and a display control circuit 171. The image data generated by the analysis processing unit 137 is sent to the display control circuit 171 by a command from the main control unit 151, and is displayed on the monitor 172 as the electromyogram in accordance with display control by the display control circuit 171.

The instructor or exerciser of the pelvic floor muscle training can understand the activity state of the transverse abdominal muscle (see FIG. 2) by observing the electromyogram displayed on the monitor 172.

### 3. Evaluation by Echo Image

As described above, the configuration required for generating the echo image includes the ultrasound sensor 201 as the biological information acquisition sensor BIS and the echo image generation units 211A to 211D. The echo image generation unit according to the present embodiment is denoted by reference numeral 211A, and the echo image generation units according to the other three embodiments described later are denoted by reference numerals 211B to 211D, respectively.

### (1) Ultrasound Sensor

As shown in FIG. 9, the ultrasound sensor 201 includes the ultrasound probe 202 in a housing (see FIG. 4) of a handy type that can be pressed against the human body 11 while being gripped with one hand. The ultrasound probe 202 includes a transducer array 203 in which a plurality of ultrasound transducers that emit ultrasound are arranged. The transducer array 203 receives the ultrasound echo that has returned after being reflected from the inside of the human body 11, and outputs the ultrasound echo as a signal of the ultrasound echo.

Each ultrasound transducer constituting the transducer array 203 has a structure (not shown) in which electrodes are provided at both ends of a piezoelectric body such as a piezoelectric ceramic, a polymer piezoelectric element, or a piezoelectric single crystal.

In the present embodiment, the ultrasound sensor 201 is pressed against the human body 11 so that the ultrasound probe 202 is brought into contact with the sensor sheet 101 attached to the region of the abdomen 12 corresponding to the transverse abdominal muscle. In a case where the transducer array 203 emits ultrasound at this position, an ultrasound echo reflected from the bladder can be acquired. In this case, since the entire sensor sheet 101 is generated using the elastomer as a material, the ultrasound emitted from the transducer array 203 is propagated through the inside of the sensor sheet 101. In addition, the ultrasound echo returning from the human body 11 as the echo is also propagated through the inside of the sensor sheet 101 and is received by the transducer array 203.

### (2) Echo Image Generation Unit

The echo image generation unit 211A includes an echo signal transmission/reception circuit 231, an ultrasound image generation unit 241, a main control unit 251, and a display control circuit 271. The main control unit 251 is configured by, for example, a microcomputer that interprets and executes a program or an integrated circuit that sequentially executes a specified process, and is responsible for controlling the echo signal transmission/reception circuit 231, the ultrasound image generation unit 241, and the display control circuit 271.

The echo signal transmission/reception circuit 231 includes a pulse generator 232 that drives the transducer array 203 of the ultrasound probe 202. The pulse generator 232 is a collection of a plurality of pulse oscillators. Each oscillator outputs a drive signal (voltage) in which a delay amount is adjusted toward electrodes of the individual ultrasound transducers constituting the transducer array 203 based on a transmission delay pattern corresponding to a control signal from the main control unit 251. When a pulsed or continuous wave voltage is applied to the electrodes of the individual ultrasound transducers of the transducer array 203, the piezoelectric body expands and contracts, and a pulsed or continuous wave ultrasound is generated from each ultrasound transducer. An ultrasound beam is formed by a composite wave of the ultrasounds.

The ultrasound emitted by the transducer array 203 is reflected from the inside of the human body 11, that is, from the bladder in the case of the present embodiment, and returns to the transducer array 203 as an ultrasound echo. The transducer array 203 outputs a signal corresponding to the received ultrasound echo.

The echo signal transmission/reception circuit 231 includes an amplifier 233, an AD converter 234, and a beam former 235.

The signal of the ultrasound echo output from the transducer array 203 is amplified by the amplifier 233, is converted into a digital signal by the AD converter 234, and is input to the beam former 235. The beam former 235 performs receive focusing processing by adding, to the digitalized signals from the individual ultrasound transducers of the transducer array 203 received from the AD converter 234, a corresponding delay. By performing the receive focusing processing, the output signals of the individual ultrasound transducers that are digitalized by the AD converter 234 are subjected to integer summation, and ultrasound image data in which the focus of the ultrasound echo is narrowed is generated.

The ultrasound image generation unit 241 includes a signal processing unit 242, a digital scan converter (DSC) 243, and an image processing unit 244 connected in series.

The signal processing unit 242 performs various types of processing on the ultrasound image data output from the beam former 235 of the echo signal transmission/reception circuit 231 to generate M-mode image data.

The DSC 243 performs raster conversion on the M-mode image data generated by the signal processing unit 242 into image data according to a scanning method of a normal television signal.

The image processing unit 244 performs various types of image processing on the image data converted by the DSC 243, and then sends the image data to the display control circuit 271 in response to a command from the main control unit 251.

The display control circuit 271 displays an image (M-mode image) of the ultrasound echo according to the image data of the ultrasound echo output from the image processing unit 244 on the monitor 272 under the control of the main control unit 251. A motion image of the bladder is displayed on the monitor 272.

As described above, the instructor or exerciser of the pelvic floor muscle training understands the activity state of the transverse abdominal muscle (see FIG. 2) by observing the electromyogram displayed on the monitor 172. Here, by referring to the motion image (M-mode image) of the bladder displayed on the monitor 272, it is possible to confirm whether or not the pelvic floor muscle is actually contracted by the pelvic floor muscle training being performed.

### 4. Biological Information Acquisition Device

In the present embodiment, as a configuration for generating the electromyogram, the sensor sheet 101 and the electromyogram generation unit 131A as the image generation unit are provided. In addition, as a configuration for generating the echo image, the ultrasound sensor 201 and the echo image generation unit 211A as the image generation unit are provided.

The electromyogram generation unit 131A generates image data of the electromyogram based on the electric signal extracted from the output end 113a of the wiring lines 113 included in the sensor sheet 101.

The echo image generation unit 211A generates image data of the ultrasound echo based on the signal of the ultrasound echo output from the ultrasound probe 202 included in the ultrasound sensor 201.

In the present embodiment, the sensor sheet 101 and the ultrasound sensor 201 are understood as the biological information acquisition sensor BIS. Then, by adding the electromyogram generation unit 131A and the echo image generation unit 211A to the biological information acquisition sensor BIS, biological information acquisition devices BIA-A to BIA-D are configured. The biological information acquisition device according to the present embodiment is denoted by reference numeral BIA-A, and the biological information acquisition devices according to the other three embodiments described later are denoted by reference numerals BIA-B to BIA-D, respectively.

### 5. Biological Information Acquisition Method

In the present embodiment, the biological information acquisition sensor BIS (the sensor sheet 101 and the ultrasound sensor 201) is applied to the exerciser performing the pelvic floor muscle training, and the activity state of the pelvic floor muscle is observed by the biological information acquisition device BIA-A (the electromyogram generation unit 131A and the echo image generation unit 211A).

In this case, the biological information acquisition method executes a first step of attaching the sensor sheet 101 to the region of the abdomen corresponding to the transverse abdominal muscle, and a second step of pressing the ultrasound sensor 201 against the abdomen 12 so that the ultrasound probe 202 is brought into contact with the sensor sheet 101 attached to the human body 11.

### (1) First Step

The sensor sheet 101 is attached to the human body 11 so that a surface of the sensor sheet 101 on the insulating layer 114 side on which the electrodes 112 are exposed comes into contact with the body. A position to which the sensor sheet 101 is attached is the region of the abdomen 12 corresponding to the transverse abdominal muscle (see FIGS. 2 and 4).

The sensor sheet 101 is configured with the sheet 111, the electrodes 112, the wiring lines 113, and the insulating layer 114, which are made of an elastomer, and has not only stretchability and flexibility but also conformability to the human body 11 (see FIGS. 5 to 7). Therefore, the sensor sheet 101 can be brought into close contact with the region of the abdomen 12 corresponding to the transverse abdominal muscle that is a measurement target of the myoelectric potential.

An electric signal of the myoelectric potential generated by the transverse abdominal muscle can be extracted from the sensor sheet 101 brought into close contact with the abdomen 12 through the electrodes 112. Therefore, the electromyogram generation unit 131A generates image data of the electromyogram indicating the activity status of the transverse abdominal muscle based on the electric signal extracted from the sensor sheet 101, and displays the electromyogram based on the generated image data on the monitor 172.

Since the transverse abdominal muscle has a cooperative contraction with the pelvic floor muscle, the activity state of the pelvic floor muscle can be estimated by referring to the electromyogram of the transverse abdominal muscle displayed on the monitor 172.

### (2) Second Step

The cooperative contraction between the pelvic floor muscle and the transverse abdominal muscle does not occur with a probability of 100% (see p. 88, “3-8-2” in Tsujino et al.). It is presumed that there is a causal relationship in the cooperative contraction between the pelvic floor muscle and the transverse abdominal muscle, but the causal relationship is not clear, and even if the causal relationship is clarified, it is difficult to convey the causal relationship in words and to feel the causal relationship. Therefore, the biological information acquisition method according to the present embodiment enables the activity of the bladder that can be regarded as the contraction movement of the pelvic floor muscle to be observed by using a method of the ultrasound echo while the activity status of the transverse abdominal muscle is observed by the electromyogram.

In the second step, in order to acquire the echo image of the bladder, the ultrasound sensor 201 is pressed against the abdomen 12 so that the ultrasound probe 202 is brought into contact with the sensor sheet 101 attached to the abdomen 12.

The echo image generation unit 211A generates image data of the ultrasound echo based on the signal of the ultrasound echo output from the ultrasound probe 202, and displays an image (M-mode image) of the ultrasound echo based on the generated image data on the monitor 272.

The motion image of the bladder is displayed on the monitor 272. Since the state of the bottom of the bladder directly reflects the activity state of the pelvic floor muscle, it is possible to confirm whether or not the pelvic floor muscle has a cooperative contraction with the transverse abdominal muscle by referring to the image of the ultrasound echo displayed on the monitor 272.

### (3) Summary

The ultrasound sensor 201 acquires the echo image of the bladder in a state where the ultrasound probe 202 is brought into contact with the sensor sheet 101 attached to the abdomen 12. This is possible because the ultrasound emitted from the transducer array 203 of the ultrasound probe 202 is propagated through the inside of the sensor sheet 101, reaches the bladder (see FIGS. 1B and 1C), and after being reflected by the bladder, is propagated again through the inside of the sensor sheet 101 to return to the transducer array 203. In other words, it can be said to be attributable to the property of the sensor sheet 101 to propagate ultrasound.

It is presumed that the property of propagating the ultrasound is a property obtained by using an elastomer, particularly a urethane-based elastomer, as the material of the sheet 111, the electrodes 112, the wiring lines 113, and the insulating layer 114, which are components of the sensor sheet 101. It is also presumed that the fact that the thicknesses of the sheet 111 and the insulating layer 114 are 50 μm or less, for example, 10 to 25 μm or less, or the electrodes 112 and the wiring lines 113 are generated by the printing method also contributes to imparting the property of propagating the ultrasound to the sensor sheet 101.

According to the present embodiment, the activity state of the pelvic floor muscle during the execution of the pelvic floor muscle exercise can be easily and accurately understood.

### 6. Another Example of Image Generation Unit

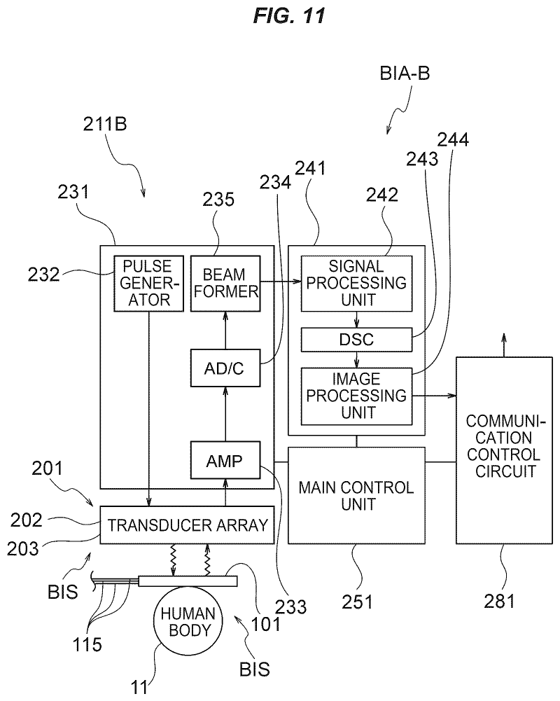

The electromyogram generation unit 131B, which is another example of the image generation unit, will be described with reference to FIG. 10, and the echo image generation unit 211B will be described with reference to FIG. 11 The same parts as the electromyogram generation unit 131A described with reference to FIG. 8 and the echo image generation unit 211A described with reference to FIG. 9 are denoted by the same reference numerals, and the description thereof will be omitted.

The biological information acquisition device BIA-B according to the present embodiment includes the electromyogram generation unit 131B and the echo image generation unit 211B.

### (1) Electromyogram Generation Unit

In the electromyogram generation unit 131B, instead of the display control circuit 171, a communication control circuit 181 is connected to the main control unit 151 and the analysis processing unit 137. The communication control circuit 181 includes a communication interface (not shown) that executes at least one of wired communication and wireless communication in accordance with a specific communication protocol. The communication control circuit 181 that receives a command from the main control unit 151 transmits and outputs video data of the electromyogram received from the analysis processing unit 137 to an external device (not shown).

The analysis processing unit 137 of the electromyogram generation unit 131B edits the image data of the electromyogram into a form of video data that is reproducible by a media player, which is software for reproducing a video. The main control unit 151 sends a control command to the analysis processing unit 137 and outputs the generated video data of the electromyogram to the communication control circuit 181. The communication control circuit 181 receives a command from the main control unit 151 and transmits and outputs the video data of the electromyogram received from the analysis processing unit 137 via wired communication or wireless communication.

In a case where the communication control circuit 181 includes an interface for wired communication, when an information device that supports a common communication protocol and that has the media player installed, such as a personal computer (not shown), is connected to the communication control circuit 181, the video data of the electromyogram generated by the analysis processing unit 137 is transmitted to the personal computer. The personal computer can reproduce the received video data of the electromyogram on the media player and display the video data on a display (not shown).

In a case where the communication control circuit 181 includes an interface for wireless communication, the video data of the electromyogram wirelessly transmitted by the communication control circuit 181 can be reproduced by an information device that supports a common communication protocol and that has the media player installed, such as a smartphone or a tablet terminal.

### (2) Echo Image Generation Unit

In the echo image generation unit 211B, instead of the display control circuit 271, a communication control circuit 281 is connected to the main control unit 251 and the image processing unit 244. The communication control circuit 281 includes a communication interface (not shown) that executes at least one of wired communication and wireless communication in accordance with a specific communication protocol. The communication control circuit 281 that receives a command from the main control unit 251 transmits and outputs video data of the ultrasound echo received from the image processing unit 244 to an external device (not shown).

The image processing unit 244 of the echo image generation unit 211B edits image data of the ultrasound echo into a form of video data that is reproducible by a media player, which is software for reproducing a video. The main control unit 251 sends a control command to the image processing unit 244 and outputs the generated video data of the ultrasound echo to the communication control circuit 281. The communication control circuit 281 receives a command from the main control unit 251 and transmits and outputs the video data of the ultrasound echo received from the image processing unit 244 via wired communication or wireless communication.

In a case where the communication control circuit 281 includes an interface for wired communication, when an information device that supports a common communication protocol and that has the media player installed, such as a personal computer (not shown), is connected to the communication control circuit 281, the video data of the ultrasound echo generated by the image processing unit 244 is transmitted to the personal computer. The personal computer can reproduce the received video data of the ultrasound echo on the media player and display the video data on a display (not shown).

In a case where the communication control circuit 281 includes an interface for wireless communication, the video data of the ultrasound echo wirelessly transmitted by the communication control circuit 281 can be reproduced by an information device that supports a common communication protocol and that has the media player installed, such as a smartphone or a tablet terminal.

### (3) Display Aspect

Regarding the electromyogram, three aspects of viewing methods, that is, viewing on the monitor 172, viewing on a wired device such as a personal computer, and viewing on a wireless device such as a smartphone or a tablet terminal, have been introduced. Regarding the ultrasound echo, three aspects of viewing methods, that is, viewing on the monitor 272, viewing on a wired device such as a personal computer, and viewing on a wireless device such as a smartphone or a tablet terminal, have been introduced. There are a total of six aspects.

These six aspects of the viewing methods can be applied in combination as appropriate.

For example, various aspects of viewing methods are allowed, such as an aspect in which the electromyogram is viewed on the monitor 172 and the ultrasound echo is viewed on the smartphone, and an aspect in which both the electromyogram and the ultrasound echo are viewed on displays of personal computers arranged side by side. In practice, the instructor or the exerciser of the pelvic floor muscle training may appropriately select a viewing aspect that is easy to see and easy to check.

### 7. Another Example of Image Generation Unit

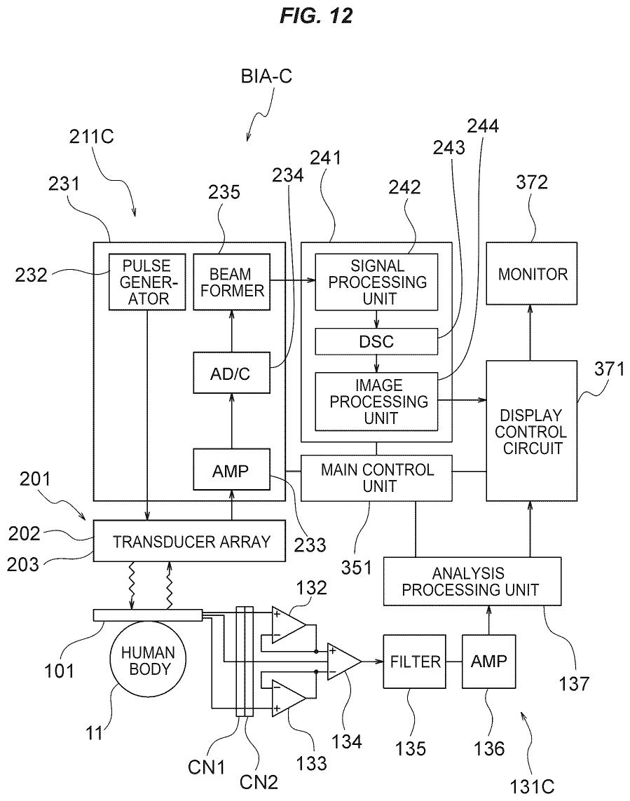

Another example (the electromyogram generation unit 131C and the echo image generation unit 211C) of the image generation unit will be described with reference to FIG. 12. The present embodiment is based on the image generation unit (the electromyogram generation unit 131A and the echo image generation unit 211A) described with reference to FIGS. 8 and 9. Therefore, the same parts as the first embodiment described with reference to FIGS. 8 and 9 are denoted by the same reference numerals, and the description thereof will be omitted.

In the present embodiment, the electromyogram generation unit 131A and the echo image generation unit 211A are integrated, and the main control units 151 and 251 are unified as a main control unit 351. In addition, the display control circuits 171 and 271 are unified as a display control circuit 371, and the monitors 172 and 272 are unified as a monitor 372.

Therefore, the image data of the electromyogram generated by the analysis processing unit 137 is sent to the display control circuit 371 by a command from the main control unit 351, and is displayed on the monitor 372 as the electromyogram in accordance with display control by the display control circuit 371. In addition, the image data (M-mode image) of the ultrasound echo generated by the image processing unit 244 is sent to the display control circuit 371 by a command from the main control unit 351, and is displayed on the monitor 372 as the ultrasound echo in accordance with display control by the display control circuit 371.

In this case, the display control circuit 371 edits the image data of the electromyogram and the image data of the ultrasound echo as integrated data on one screen in which time axes are synchronized in response to a command from the main control unit 351. The electromyogram and the ultrasound echo are displayed on the monitor 372, for example, in an up-down direction with the same time axis.

Therefore, since the electromyogram and the ultrasound echo are shown on one screen of the single monitor 372 with the same time axis, it is possible to easily check whether or not the pelvic floor muscle is contracted together with the transverse abdominal muscle.

### 8. Still Another Example of Image Generation Unit

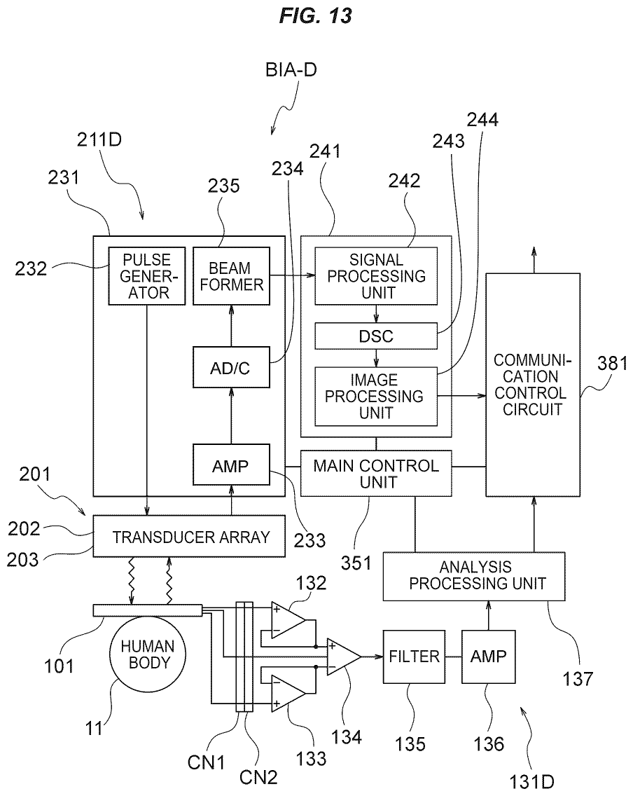

Still another example (the electromyogram generation unit 131D and the echo image generation unit 211D) of the image generation unit will be described with reference to FIG. 13. The present embodiment is based on the image generation unit (the electromyogram generation unit 131B and the echo image generation unit 211B) described with reference to FIGS. 10 and 11. Therefore, the same parts as the second embodiment described with reference to FIGS. 10 and 11 are denoted by the same reference numerals, and the description thereof will be omitted.

In the present embodiment, the electromyogram generation unit 131B and the echo image generation unit 211B are integrated, and the main control units 151 and 251 are unified as the main control unit 351. In addition, the communication control circuits 181 and 281 are unified as a communication control circuit 381.

Therefore, the image data of the electromyogram generated by the analysis processing unit 137 is sent to the communication control circuit 381 by a command from the main control unit 351, and is transmitted and output to an external device (not shown) as video data of the electromyogram that can be viewed by the media player. In addition, the image data (M-mode image) of the ultrasound echo generated by the image processing unit 244 is sent to the communication control circuit 381 by a command from the main control unit 351, and is transmitted and output to an external device (not shown) as video data of the ultrasound echo that can be viewed by the media player.

In this case, the communication control circuit 381 edits the image data of the electromyogram and the image data of the ultrasound echo as integrated data on one screen in which time axes are synchronized in response to a command from the main control unit 351.

In the external device that has received the integrated data, such as a personal computer or a smartphone, the electromyogram and the ultrasound echo are displayed, for example, in an up-down direction with the same time axis by the media player.

Therefore, since the electromyogram and the ultrasound echo are shown on a single display screen of the external device with the same time axis, it is possible to easily check whether or not the pelvic floor muscle is contracted together with the transverse abdominal muscle.

### 9. Modification Examples

In practice, various modifications and changes are allowed.

For example, for the sensor sheet 101, a specific shape, various numerical values, a manufacturing method, and the like are shown for each part such as the sheet 111, the electrodes 112, and the wiring lines 113, but these are merely one embodiment, and various modifications and changes may be made in practice.

In addition, in the biological information acquisition devices BIA-A to BIA-D, an example is shown in which the sensor sheet 101 as the biological information acquisition sensor BIS and the electromyogram generation units 131A to 131D are connected by the connectors CN1 and CN2, but the connectors CN1 and CN2 are not necessarily required in practice. For example, the connection line 115 extending from the sensor sheet 101 may be directly connected to the two buffer amplifiers 132 and 133 and the one differential amplifier 134.

In addition, the electromyogram generation units 131a to 131d may be accommodated in one housing as a whole or may be separately accommodated in a plurality of housings.

The same applies to the echo image generation units 211A to 211D. For example, the ultrasound sensor 201 may accommodate only the ultrasound probe 202 in the housing as shown in FIG. 4, or may also accommodate other circuits such as the echo signal transmission/reception circuit 231 and the signal processing unit 242, and either configuration may be adopted.

In addition, in practice, any change or modification is allowed.

### EXAMPLES

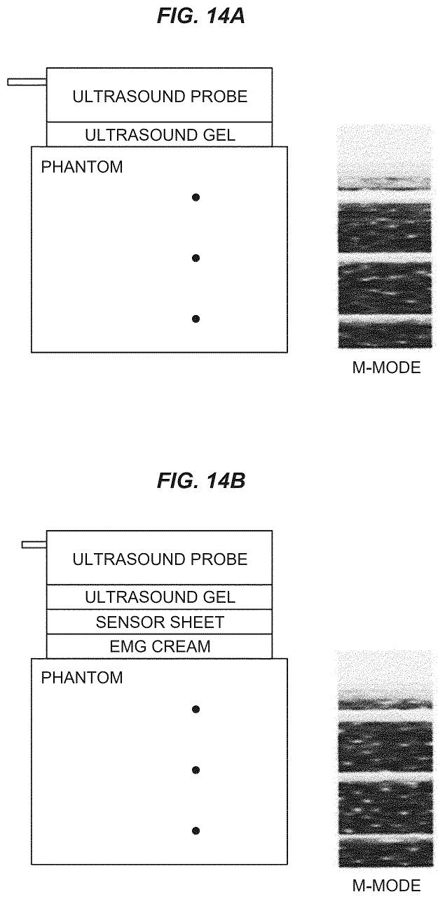

The inventors of the present application have experimentally verified whether or not the image of the ultrasound echo that reflects the movement of the bladder can be normally obtained even from above the sensor sheet by propagating the ultrasound through the sensor sheet. The content and the results of the experiment will be reported based on FIGS. 14A to 18.

### 1. Experiment Using Phantom

### (1) Experimental Equipment

As shown in FIGS. 14A and 14B, in the experiment, a phantom was used, and an image (M-mode image) of an ultrasound echo in the phantom was acquired by an ultrasound probe. In this case, a case without the sensor sheet (see FIG. 14A) and a case with the sensor sheet (see FIG. 14B) were compared to verify the extent to which the sensor sheet affects the image of the ultrasound echo.

The phantom used in the experiment was an ultrasound evaluation phantom US-2 manufactured by Kyoto Kagaku Co., Ltd. Three wires were incorporated in the phantom. In FIGS. 14A and 14B, the three wires are displayed as black dots arranged in a vertical line.

The sensor sheet, that is, a bioelectric potential sensor used was a stretchable flexible printed circuit (FPC) manufactured by Mektec Corporation.

The ultrasound probe used was a convex type ultrasound probe “VSCAN Air (product name)” manufactured by GE Healthcare Japan.

In addition, in the experiment, an ultrasound gel and an EMG cream were also used. The ultrasound gel used was an ultrasound gel, “F JELLY PLUS (product name)”, manufactured by FUJIFILM Corporation. The EMG cream used was an EMG cream manufactured by Kenz.

### (2) Experimental Results

FIG. 14A shows an example of the experiment (comparative experiment) in which the sensor sheet was not used.

In this experimental example, the ultrasound gel was applied to the phantom, and the ultrasound probe was brought into contact with the ultrasound gel to irradiate the phantom with ultrasound. In FIG. 14A, an image drawn above the text “M-mode” is an M-mode image at a certain moment of the ultrasound echo acquired by the ultrasound probe. It can be seen that the three wires are displayed in white.

FIG. 14B shows an example of an experiment (verification experiment) in which the sensor sheet was used.

In this experimental example, the EMG cream was applied to the phantom, and the sensor sheet was attached thereto. The ultrasound gel was applied to the sensor sheet, the ultrasound probe was brought into contact with the ultrasound gel to irradiate the phantom with ultrasound. In FIG. 14B, an image drawn above the text “M-mode” is an M-mode image at a certain moment of the ultrasound echo acquired by the ultrasound probe. It can be seen that the three wires are displayed in white.

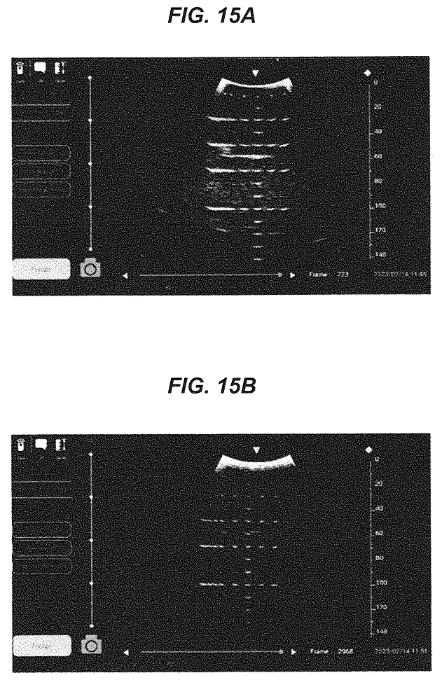

FIG. 15A is a schematic view showing an echo image obtained in the comparative experiment. A certain moment is captured from the M-mode image without the sensor sheet.

FIG. 15B is a schematic view showing an echo image obtained in the verification experiment. A certain moment is captured from the M-mode image in which the ultrasound was propagated through the sensor sheet.

When the image of FIG. 15A was compared with the image of FIG. 15B, it was confirmed that, although the image of the ultrasound echo of the latter is overall darker than the image of the former, there was no significant decrease in quality, and an image sufficient for visual evaluation of the inside of a living body could be obtained.

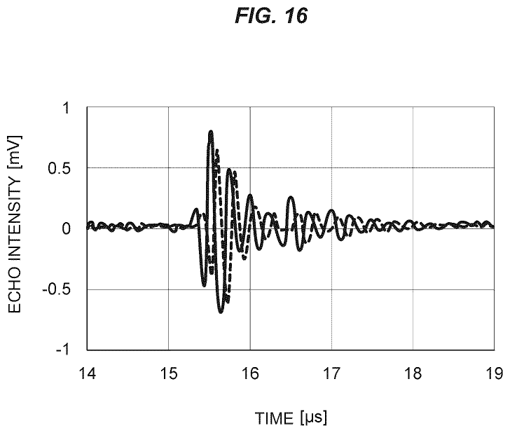

FIG. 16 is a graph showing waveforms of echo intensities obtained in the experiments shown in FIGS. 14A and 14B. The echo intensity is a voltage (mV) output from the transducer array of the ultrasound probe.

In FIG. 16, the echo intensity in the comparative experiment (see FIG. 14A) in which the sensor sheet was not used is indicated by a solid line. The echo intensity in the verification experiment (see FIG. 14B) in which the sensor sheet was used is indicated by a dotted line.

Referring to the graph shown in FIG. 16, it can be seen that, even when the sensor sheet is used, there is no significant difference in the echo intensity compared to when the sensor sheet is not used. Therefore, it was proven that even from the viewpoint of the echo intensity, an image sufficient for visual evaluation of the inside of a living body can be obtained from the signal of the ultrasound echo acquired through the sensor sheet,.

### 2. Experiment Using Human Body

### (1) Experimental Equipment

As in the embodiment, electromyogram measurement of the transverse abdominal muscle (see FIG. 2) and echo measurement of the abdomen were performed simultaneously. A sensor sheet, an ultrasound probe, an ultrasound gel, and an EMG cream used in the experiment were the same as those in the above-described section “1. Experiment Using Phantom”.

In the experiment using a human body, an experiment (comparative experiment) in which the sensor sheet was not used and an experiment (verification experiment) in which the sensor sheet was used were conducted.

In the comparative experiment, the ultrasound gel was applied to a region of the abdomen corresponding to the transverse abdominal muscle, and the ultrasound probe was brought into contact with the ultrasound gel to acquire a signal of an ultrasound echo.

In the verification experiment, the EMG cream was applied to the region of the abdomen corresponding to the transverse abdominal muscle, and the sensor sheet was attached thereto. Then, the ultrasound gel was applied to the sensor sheet, and the ultrasound probe was brought into contact with the ultrasound gel to acquire the ultrasound echo. Here, a signal of a myoelectric potential was acquired from the sensor sheet.

### (2) Experimental Results

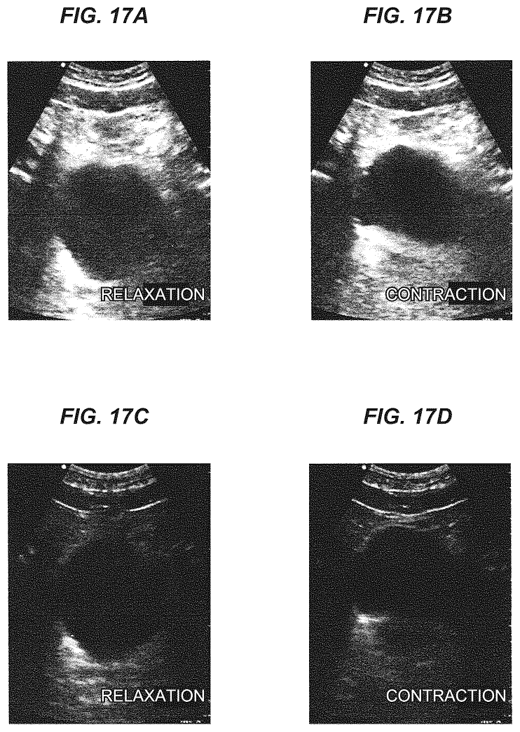

FIGS. 17A and 17B are echo images obtained in the comparative experiment, each showing a certain moment captured from the M-mode image. FIG. 17A is an image at the time of muscle relaxation, and FIG. 17B is an image at the time of muscle contraction. A black portion seen at the center is the bladder, and by observing the state of the bottom of the bladder, it is possible to understand the movement state of the pelvic floor muscle located below the bladder.

FIGS. 17C and 17D are echo images obtained in the verification experiment, each showing a certain moment captured from the M-mode image. FIG. 17C is an image at the time of muscle relaxation, and FIG. 17D is an image at the time of muscle contraction. Even the echo images obtained in the verification experiment clearly show the bladder changing in shape and the displacement state of the bottom of the bladder. Therefore, it is possible to understand the movement state of the pelvic floor muscle located below the bottom of the bladder.

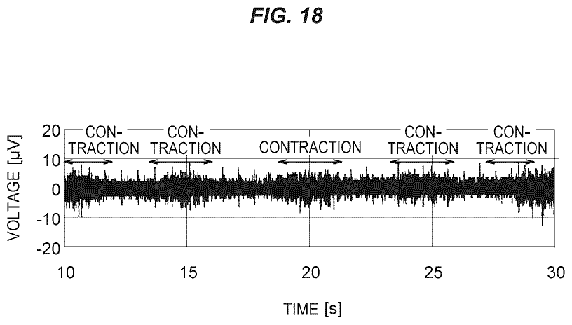

FIG. 18 is a graph showing contraction timings of the muscle shown in FIGS. 17C and 17D superimposed on an image of an electromyogram obtained in the verification experiment. The horizontal axis represents time (10 to 30 seconds), and the vertical axis represents voltage values (−20 to 20 μV) indicating the magnitude of the myoelectric potential.

As shown in FIG. 18, the voltage values fluctuate in accordance to the increase or decrease in the myoelectric potential acquired from the sensor sheet attached to the region of the abdomen corresponding to the transverse abdominal muscle. An interval in which the voltage value is large is an interval in which contraction of the transverse abdominal muscle occurs. Muscle contraction of the transverse abdominal muscle occurs generally in intervals of 10 to 11.5 seconds, 13.5 to 16 seconds, 19 to 20 seconds, 23.5 to 26 seconds, and 28.5 to 30 seconds.

In FIG. 18, the intervals indicated by double arrows and labelled “contraction” correspond to intervals in which the displacement of the bottom of the bladder, that is, the contraction of the pelvic floor muscle understood from the image of the ultrasound echo occurs. From the graph shown in FIG. 18, it can be confirmed that the contraction of the transverse abdominal muscle and the contraction of the pelvic floor muscle occur at approximately the same timing, except for the intervals of 28.5 to 30 seconds. At this timing, the pelvic floor muscle training is being correctly performed.

Contrary to this, the voltage value indicating the contraction of the transverse abdominal muscle increases momentarily at around 27 seconds, and the contraction occurs in the pelvic floor muscle during the interval of 27 to 29 seconds. However, during this period, until about 28.5 seconds, the voltage value indicating the contraction of the transverse abdominal muscle decreases. After 28.5 seconds, the voltage value increases, but at 29 seconds, the contraction of the pelvic floor muscle is relieved. From such a measurement result, it is estimated that the contraction of the pelvic floor muscle does not occur as intended by the exerciser of the pelvic floor muscle training in the interval of 27 to 30 seconds.

Obviously, numerous modifications and variations of the present invention are possible in light of the above teachings. It is therefore to be understood that within the scope of the appended claims, the invention may be practiced otherwise than as specifically described herein.

What is claimed is:

1. A biological information acquisition sensor comprising:
a sensor sheet that is attached to a human body and acquires an electric signal generated by a muscle; and
an ultrasound sensor including an ultrasound probe configured to be pressed on the sensor sheet attached to the human body, the ultrasound sensor receiving a signal of an ultrasound echo from the human body,
wherein the sensor sheet includes
a sheet made of an elastomer,
a plurality of electrodes provided on the sheet and made of an elastomer to which conductivity is imparted,
a plurality of wiring lines that are respectively connected to the electrodes, provided on the sheet, and made of an elastomer to which conductivity is imparted, and
an insulating layer made of an elastomer and fixed to the sheet to cover the wiring lines while leaving portions of the electrodes.

2. The biological information acquisition sensor according to claim 1,
wherein the sensor sheet configured to attach to a region of an abdomen corresponding to a transverse abdominal muscle and receives an electric signal generated by the transverse abdominal muscle through the electrodes, and
the ultrasound sensor receives a signal of an ultrasound echo including a bottom of a bladder.

3. The biological information acquisition sensor according to claim 1,
wherein the elastomer used as a material of the sheet, the electrodes, the wiring lines, and the insulating layer is a urethane-based elastomer.

4. The biological information acquisition sensor according to claim 1,
wherein a thickness of the sheet is 50 μm or less.

5. A biological information acquisition device comprising:
the biological information acquisition sensor according to claim 1;
an electromyogram generation unit that generates image data of an electromyogram based on an electric signal extracted from an output end of the wiring line included in the sensor sheet; and
an echo image generation unit that generates, based on a signal of an ultrasound echo output by the ultrasound probe included in the ultrasound sensor, image data of the ultrasound echo.

6. A biological information acquisition device comprising:
the biological information acquisition sensor according to claim 1; and
an image generation unit that executes image generation processing based on an output signal of the biological information acquisition sensor,
wherein the image generation unit generates image data of an electromyogram based on an electric signal extracted from an output end of the wiring line included in the sensor sheet, and generates, based on a signal of an ultrasound echo output by the ultrasound probe included in the ultrasound sensor, image data of the ultrasound echo.

7. The biological information acquisition device according to claim 6,
wherein the image generation unit edits the image data of the electromyogram and the image data of the ultrasound echo as integrated data on one screen in which time axes are synchronized.

8. A biological information acquisition method using the biological information acquisition sensor according to claim 1, the biological information acquisition method comprising:
attaching the sensor sheet to a region of an abdomen corresponding to a transverse abdominal muscle; and
pressing the ultrasound sensor against the abdomen so that the ultrasound probe is brought into contact with the sensor sheet attached to the human body.

9. The biological information acquisition method according to claim 8,
wherein the elastomer used as a material of the sheet, the electrodes, the wiring lines, and the insulating layer is a urethane-based elastomer.

10. The biological information acquisition method according to claim 8,
wherein a thickness of the sheet is 50 μm or less.

11. The biological information acquisition method according to claim 8, further comprising:
generating image data of an electromyogram based on an electric signal extracted from an output end of the wiring line included in the sensor sheet, and
generating image data of the ultrasound echo based on a signal of an ultrasound echo output by the ultrasound probe included in the ultrasound sensor.

12. The biological information acquisition method according to claim 11,
wherein an image is displayed on a monitor based on at least one of the image data of the electromyogram and the image data of the ultrasound echo.

13. The biological information acquisition method according to claim 11, further comprising:
editing at least one of the image data of the electromyogram and the image data of the ultrasound echo as video data that is reproducible by a media player, and
transmitting and outputting the video data externally.

14. The biological information acquisition method according to claim 11,
wherein the image data of the electromyogram and the image data of the ultrasound echo are edited as integrated data on one screen in which time axes are synchronized.

15. The biological information acquisition method according to claim 14,
wherein an image is displayed on a monitor based on the integrated data.

16. The biological information acquisition method according to claim 14,
wherein the integrated data is edited as video data that is reproducible by a media player, and
the video data is externally transmitted and output.
